(19) United States
(12) Patent Application Publication
(10) Pub. No.: US 2002/0032153 A1
(43) Pub. Date: Mar. 14, 2002

(54) Methods and compositions for the treatment and prevention of erectile dysfunction

(76) Inventor: Martha Jo Whitehouse, San Francisco, CA (US)

Correspondence Address: Chiron Corporation, Intellectual Property, P.O. Box 8097, Emeryville, CA, 94662-8097, US

(21) Appl. No.: 09/802,365

(22) Filed: Mar. 9, 2001

Related U.S. Application Data
(60) Provisional application No. 60/188,480, filed on Mar. 10, 2000.
(60) Provisional application No. 60/203,415, filed on May 11, 2000.

(51) Int. Cl. A61K 38/18

(52) U.S. Cl. 514/12

(57) ABSTRACT

Compositions and methods for improving erectile function in a patient are provided. Compositions comprise one or more angiogenic agents or growth factors. Such agents or growth factors are administered in therapeutically effective amounts to treat or prevent erectile dysfunction. Pharmaceutical compositions comprising a therapeutically effective amount of at least one angiogenic agent or growth factor and a pharmaceutically acceptable carrier are also provided.

The methods of the invention to improve erectile function and treat erectile dysfunction comprise administering at least a single unit dose of a pharmaceutical composition comprising the angiogenic agent or growth factor, generally at a local target site in the patient. It is recognized that increased benefits may result from multiple dosing, including intermittent dosing.

### CROSS-REFERENCE TO RELATED APPLICATIONS

This application claims the benefit of U.S. Provisional Application Ser. No. 60/188,480, filed Mar. 10, 2000, and No. 60/203,415, filed May 11, 2000, both entitled “Methods and Compositions for the Treatment and Prevention of Erectile Dysfunction,” herein incorporated by reference in their entirety.

### FIELD OF THE INVENTION

The invention relates to methods and pharmaceutical compositions for treating erectile dysfunction, particularly for the administration of angiogenic agents or growth factors that promote endothelial cell proliferation, endothelial cell function, and angiogenesis to treat and prevent erectile dysfunction.

### BACKGROUND OF THE INVENTION

Erectile dysfunction, or impotence, is the consistent inability to achieve or sustain an erection sufficient for sexual intercourse. Reportedly, 10 to 30 million men in America show symptoms of erectile dysfunction (Shabsigh et al. (1988) Urology 32:83-90). The erection process begins when impulses from the brain and local nerves cause muscles in the penis to relax. When these muscles are relaxed, blood flows into the corpora cavernosa (the two chambers that run the length of the penis), causing the penis, to expand. The tunica albuginea is a thick membrane that surrounds the corpora cavernosa to help trap the blood causing the penis to remain erect for sexual intercourse. After ejaculation, the muscles contract allowing the blood to flow out of the penis.

Since achieving an erection requires a series of events, any interruption of the sequence can cause erectile dysfunction. Erectile dysfunction can be the result of psychological disturbances, physiological abnormalities, vascular abnormalities, neurological disturbances, hormonal deficiencies, or a combination of any of these. Thus, erectile dysfunction has been defined as psychogenic, neurogenic, hormonal, vascular, cavernosal, drug induced, or a combination of these etiologic elements. Generally, the incidence of erectile dysfunction increases with increasing age. A majority of patients with erectile dysfunction suffer from aging-related changes of the neurovascular supply of the penis and the pelvic ganglia (Lue (1992) “Physiology of Penile Erection and Pathophysiology of Impotence,” in Campbell's Urology, eds. Walsh et al. (6^{th} ed., W. B. Saunders), pp. 707-728). Some of the most common diseases affecting blood flow to the penis include heart disease, atherosclerosis, high blood pressure, diabetes, chronic alcoholism, liver failure, elevated cholesterol level, and the like. Some medications prescribed as treatment for these diseases also can interfere with the impulses from the brain and nerves that tell the muscles to relax, resulting in erectile dysfunction. Hormonal abnormalities, such as a lack of testosterone, also contribute to erectile dysfunction. Additionally, neurological disorders, such as spinal cord injury and multiple sclerosis, can also disrupt the process and cause erectile dysfunction.

Various methods for the treatment of erectile dysfunction have been suggested. Treatment options include oral drug therapy, vacuum constriction devices, penile injection therapy, intraurethral drug therapy, and surgery, including penile implants. External devices include tourniquets as described in U.S. Pat. No. 2,818,855. Erection-effecting and enhancing drugs have also been utilized. See, for example, U.S. Pat. No. 4,127,118. Topical agents have also been applied for the treatment of erectile dysfunction. See, U.S. Pat. Nos. 4,801,587 and 5,256,652.

At the present time, sildenafil (Viagra) is the only oral drug to successfully treat erectile dysfunction in clinic studies. Sildenafil works by increasing the concentration of cyclic guanosine monophosphate in the penis. Sildenafil causes the blood vessels to dilate, which increases the blood flow to the penis. However, sildenafil is not recommended to men who have angina or coronary artery disease. Furthermore, side effects have been associated with sildenafil including priapism, a rare but serious side effect that can cause an erection to last for more than four hours.

In rare instances, surgery is recommended for reconstruction of the arteries. However, men with atherosclerosis are usually not suitable candidates. Surgery to reconstruct arteries and increase blood flow to the penis generally has a higher success rate with young men who have had a specific injury, such as a straddle injury or pelvic fracture. During surgery, the damaged artery or arteries are by-passed. However, as with most surgeries, there is risk of infection and high cost associated with the procedure.

Erectile dysfunction remains a widespread problem with many men. Accordingly, there is needed a successful therapy for the treatment of erectile dysfunction.

### SUMMARY OF THE INVENTION

Compositions and methods for improving erectile function in a patient are provided. Compositions comprise angiogenic agents or growth factors. Such agents or factors are administered in therapeutically effective amounts to treat or prevent erectile dysfunction. Pharmaceutical compositions comprising an effective amount of at least one angiogenic agent or growth factor and a pharmaceutically acceptable carrier are also provided.

The methods of the invention comprise administering at least a single unit dose of the angiogenic agent or growth factor, generally locally in the vessels supplying the penis, as well as in the groin or leg of the patient. It is recognized that increased benefits to erectile function may result from multiple dosing, including intermittent dosing.

### DETAILED DESCRIPTION OF THE INVENTION

Compositions and methods for improving erectile function in a patient are provided. Thus, the compositions and methods are useful in the treatment and prevention of erectile dysfunction. The methods of the invention utilize angiogenic agents or growth factors to encourage endothelial cell proliferation, to restore endothelial cell function, and to promote angiogenesis, particularly to promote blood flow to the penis. Angiogenesis occurs when pre-existing vessels send out capillary buds or sprouts to produce new vessels. By “angiogenesis” is intended the formation of new blood vessels, ranging in size from capillaries to arterioles, which act as collaterals in circulation. Additionally, angiogenesis involves the proliferation of endothelial cells. Angiogenesis or the formation of new blood vessels is an important process, critical to the formation of collateral circulation.

The endothelium of the corpus cavernosum plays an important role in the physiology of erection. Endothelial cells synthesize and release constricting and relaxing factors, including endothelin and nitric oxide. Endothelin, which is a potent vasoconstrictor, is released preferentially to the basal side of endothelial cells. Endothelin may play a role in the regulation of tone in penile vascular and cavernous tissues. Nitric oxide is a powerful myorelaxant agent and serves as the main proerectile neuromediator. Other proerectile mediators, such as acetylcholine, CGRP, or substance P, act via endothelial cells by promoting the synthesis and release of nitric oxide by these cells.

While the invention is not bound by any particular mechanism of action, the angiogenic agents of the invention act to restore endothelial cell function. Additionally, they may act to promote angiogenesis.

The angiogenic agents or growth factors of the invention include natural, recombinant, and modified or variant forms of growth factors and related molecules that are able to promote endothelial and smooth muscle cell proliferation, thereby contributing to endothelial function and potentially the formation of new blood vessels. Growth factors useful in the practice of the invention include fibroblast growth factor (FGF), epidermal growth factor (EGF), platelet-derived growth factor (PDGF), vascular endothelial growth factor (VEGF), tissue growth factor (TGF), particularly TGF-β, and the like. It is recognized that compositions of the invention may comprise one or more of the therapeutic agents as well as variants, analogues, modifications, and fragments thereof.

The compositions of the invention are administered intra-arterially, intra-venously, intramuscularly (IM), transmurally, by intracavernosal injection, intraurethral patch/pellets, and the like, to a patient in need thereof. By “transmural” administration is intended localized delivery of the composition into the blood vessel or body lumen wall including the neointimal, intimal, medial, adventitial, and perivascular spaces, particularly adjacent to the target site. By “target site” is intended the area surrounding or immediately surrounding the blood flow into the penis including, but not limited to, the vessels supplying the penis. Additionally, the target site includes the groin, leg, particularly thigh, and the like. In this manner, the agents of the invention may be administered on, around, or in such vessels. Intra-arterial administration involves injection or delivery of the compositions of the invention into at least one artery, particularly into the artery feeding or supplying blood to the penis. For example, in one embodiment, compositions of the invention are administered in through the femoral near the aorta.

Delivery of the compositions according to the methods of the invention may be accomplished through a variety of known intravascular drug delivery systems. Such delivery systems include intravascular catheter delivery systems. A variety of catheter systems useful for the direct transmural infusion of angiogenic factors into the blood vessel wall are well known in the art. Balloon catheters having expandable distal ends capable of engaging the inner wall of a blood vessel and infusing an angiogenic agent or growth factor directly therein are well described in the patent literature. See, for example, U.S. Pat. Nos. 5,318,531; 5,304,121; 5,295,962; 5,286,254; 5,254,089; 5,213,576; 5,197,946; 5,087,244; 5,049,132; 5,021,044; 4,994,033; and 4,824,436. Catheters having spaced-apart or helical balloons for expansion within the lumen of a blood vessel and delivery of a therapeutic agent to the resulting isolated treatment site are described in U.S. Pat. Nos. 5,279,546; 5,226,888; 5,181,911; 4,824,436; and 4,636,195. Non-balloon drug delivery catheters are described in U.S. Pat. Nos. 5,180,366; 5,112,305; and 5,021,044; and PCT Publication WO 92/11890. Ultrasonically assisted drug delivery catheters phonophoresis devices) are described in U.S. Pat. Nos. 5,362,309; 5,318,014; and 5,315,998. Other iontophoresis and phonophoresis drug delivery catheters are described in U.S. Pat. Nos. 5,304,120; 5,282,785; and 5,267,985. Finally, sleeve catheters having drug delivery lumens intended for use in combination with conventional angioplasty balloon catheters are described in U.S. Pat. Nos. 5,364,356 and 5,336,178. All of these references are herein incorporated by reference.

The compositions of the invention provide a safe and therapeutically effective amount of an angiogenic agent or growth factor to improve erectile function and to treat erectile dysfunction. By “safe and therapeutically effective amount” is intended an amount of an angiogenic agent or growth factor, or angiogenically or biologically active variant or fragment thereof, that when administered in accordance with the invention, is free from major complications that cannot be medically managed, and that provides for objective improvement in patients having symptoms of erectile dysfunction. It is recognized that the therapeutically effective amount may vary from patient to patient depending upon age, weight, severity of symptoms, health, physical condition, and the like. Other factors include the mode of administration and the respective amount of angiogenic agent or agents included in the pharmaceutical composition. Typically, a therapeutically effective amount of an angiogenic agent or growth factor of the invention comprises about 0.01 μg/kg 5 mg/kg of the agent or factor, preferably about 0.05 μg/kg to about 1000 μg/kg, more preferably about 0.1 μg/kg to about 500 μg/kg. For example for FGF, a therapeutically effective amount may range from about 0.05 μg/kg to about 1000 μg/kg, preferably about 0.1 μg/kg to about 100 μg/kg, more preferably about 0.15 μg/kg to about 75 μg/kg, even more preferably about 0.2 μg/kg to about 48 μg/kg, more preferably still about 0.5 μg/kg to about 30 μg/kg.

As indicated, the compositions and methods of the invention are useful for treating or preventing erectile dysfunction. Further, the methods can be utilized to improve erectile function. In this manner, the desired therapeutic response will be increased erectile function in the patient as measured by the ability of the patient to achieve and sustain an erection.

The pharmaceutical compositions of the invention will be delivered for a time sufficient to achieve the desired physiological effect, i.e., improved endothelial cell function, endothelial cell proliferation, and/or angiogenesis, the promotion of blood vessel growth in tissue surrounding the target site in the blood vessel. The compositions may be administered as a single bolus released over a very short time, but will more usually be delivered as an infusion of pharmaceutical formulation over a period of time. It is recognized that any means for administration are encompassed including sustained release and other routes of administration. The total amount of time may vary depending on the delivery rate and drug concentration in the composition being delivered. Generally, the time of administration may vary from 1 second to about 24 hours, more usually from about 3 seconds to about 1 hour, specifically from about 5 seconds to about 30 minutes. Generally, for infusion the time may vary from about 5 minutes to about 10 minutes, to about 20 minutes, to about 30 minutes. When administered in accordance with the methods of the invention, these compositions provide the patient with a safe and therapeutically efficacious treatment for erectile dysfunction that lasts at least 3 months, generally 6 months, up to 12 months, before a further treatment is needed.

As indicated, the angiogenic agents of the invention include growth factors and related molecules that are able to promote endothelial and/or smooth muscle cell proliferation. Growth factors useful in the practice of the invention include fibroblast growth factors (FGF), epidermal growth factor (EGF), platelet-derived growth factor (PDGF), vascular endothelial growth factor (VEGF), tissue growth factor (TGF), particularly TGF-β, and the like. Such factors as well as variants and fragments thereof are known in the art. For example, for FGF, see U.S. Pat. Nos. 5,989,866; 5,925,528; 5,874,254; 5,817,485; 5,714,458; 5,656,458; 5,604,293; 5,576,288; 5,514,566; 5,482,929; 5,464,943; and 5,439,818; for EGF, see U.S. Pat. Nos. 5,547.935; 5,753,622; 5,705,477; and 5,916,769; for PDGF, see U.S. Pat. Nos. 5,605,816; 5,516,896; 5,512,545; 5,759,815; 5,723,594; 5,705,484; 6,018,026; 6,004,929; 6,001,802; 5,968,778; 5,935,819; and 5889,149; for VEGF, see U.S. Pat. Nos. 6,020,473; 6,013,780; 5,935,820; 5,928,939; 5,859,228; 5,840,693; 5,830,879; 5,785,965; 5,641,756; and 5,607,918; for TGF, see U.S. Pat. Nos. 5,994,094; 5,969,099; 5,807,713;, 5,801,231; 5,780,436; 5,661,127; 5,658,883; and 5,482,851; all of which are herein incorporated by reference.

The angiogenic agent to be administered can be from any animal species including, but not limited to, avian, canine, bovine, porcine, equine, and human. Generally, the agent is from a mammalian species. The agent may be in the native, recombinantly produced, or chemically synthesized forms as outlined below.

Biologically active variants of the angiogenic agents or growth factors are also encompassed by the methods of the present invention. Such variants should retain angiogenic activities or biological activities that promote endothelial cell proliferation and/or restoration of endothelial cell function. The agents or growth factors and variants thereof may be measured for angiogenic or biological activity using standard bioassays. Representative assays include known radioreceptor assays using placental membranes (see, e.g., U.S. Pat. No. 5,324,639; Hall et al. (1974) J. Clin. Endocrinol. and Metab. 39:973-976; and Marshall et al. (1974) J. Clin. Endocrinol. and Metab. 39:283-292). Additional assays include mitogenic activity as determined in an in vitro assay of endothelial cell proliferation. This activity is preferably determined in a human umbilical vein endothelial (HUVE) cell-based assay, as described, for example, in any of the following publications: Gospodarowicz et al. (1989) Proc. Natl. Acad. Sci. USA 87:7311-7315; Ferrara and Henzel (1989) Biochem. Biophys. Res. Commun. 161:851-858; Conn et al. (1990) Proc. Natl. Acad. Sci. USA 87:1323-1327; Soker et al. Cell 92:735-745; Waltenberger et al. (1994) J. Biol. Chem. 269:26988-26995; Siemmeister et al. (1996) Biochem. Biophys. Res. Commun. 222:249-255; Fiebich et al. (1993) Eur. J. Biochem. 211:19-26; Cohen et al. (1993) Growth Factors 7:131-138. A further biological activity is involvement in angiogenesis and/or vascular remodeling, which can be tested, for example, in the corneal pocket angiogenesis assay as described in Connolly et al. (1989) J. Clin. Invest. 84:1470-1478 and Lobb et al. (1985) Biochemistry 24:4969-4973; the endothelial cell tube formation assay, as described for example in Pepper et al. (1992) Biochem. Biophys. Res. Commun. 189:824-831; Goto et al. (1993) Lab. Invest. 69:508-517; or Koolwijk et al. (1996) Cell Biol. 132:1177-1188; the chick chorioallantoic membrane (CAM) angiogenesis assay as described for example in Pluet et al. (1989) EMBO. J. 8:3801-3806; the endothelial cell mitogenesis assay as described in Bohlen et al. (1984) Proc. Natl. Acad. Sci. USA 81:5364-5368; Presta et al. (1986) Mol. Gen. Biol. 6:4060-4066; Klagsbrun and Shing (1985) Proc. Natl. Acad. Sci. USA 82:805-809; Gosodarowicz et al. (1985) J. Cell. Physiol. 122:323-332; or the endothelial cell migration assay as described in Moscatelli et al. (1986) Proc. Natl. Acad. Sci. USA 83:2091-2095; and Presta et al. (1986) Mol. and Cell. Biol. 6:4060-4066; all of which are herein incorporated by reference. It is recognized that one or more of the assays may be used. Preferably, the variant has at least the same activity as the native molecule.

Suitable biologically active variants can be fragments, analogues, and derivatives. By “fragment” is intended a protein consisting of only a part of the intact angiogenic agent sequence and structure, and can be a C-terminal deletion or N-terminal deletion. By “analogues” is intended analogues of either an angiogenic agent or fragment that comprise a native sequence and structure having one or more amino acid substitutions, insertions, or deletions. Peptides having one or more peptoids (peptide mimics) are also encompassed by the term analogue. By “derivatives” is intended any suitable modification of angiogenic agents, fragments, or their respective analogues, such as glycosylation, phosphorylation, or other addition of foreign moieties, so long as the angiogenic activity is retained. Methods for making fragments, analogues, and derivatives are available in the art. See generally U.S. Pat. Nos. 4,738,921, 5,158,875, and 5,077,276; International Publication Nos. WO 85/00831, WO 92/04363, WO 87/01038, and WO 89/05822; and European Patent Nos. EP 135094, EP 123228, and EP 128733; herein incorporated by reference.

Variants of the invention will generally have at least 70%, preferably at least 80%, more preferably about 90% to 95% or more, and most preferably about 98% or more amino acid sequence identity to the amino acid sequence of the reference molecule. By “sequence identity” is intended the same amino acid residues are found within the variant and the reference molecule when a specified, contiguous segment of the amino acid sequence of the variant is aligned and compared to the amino acid sequence of the reference molecule, which serves as the basis for comparison. Thus, for example, where the reference molecule is the 146-residue human FGF-2, a biologically active variant thereof will generally have at least 70%, preferably at least 80%, more preferably about 90% to 95% or more, most preferably about 98% or more sequence identify to the full-length amino acid sequence set forth in SEQ ID NO:4.

A polypeptide that is a biologically active variant of a native FGF of interest may differ from the native FGF sequence by as few as 1-15 amino acids, as few as 1-10, such as 6-10, as few as 5, as few as 4, 3, 2, or even 1 amino acid residue. The percentage sequence identity between two amino acid sequences is calculated by determining the number of positions at which the identical amino acid residue occurs in both sequences to yield the number of matched positions, dividing the number of matched positions by the total number of positions in the segment undergoing comparison to the reference molecule, and multiplying the result by 100 to yield the percentage of sequence identity.

For purposes of optimal alignment of the two sequences, the contiguous segment of the amino acid sequence of the variant polypeptide may have additional amino acid residues or deleted amino acid residues with respect to the amino acid sequence of the reference FGF molecule. The contiguous segment used for comparison to the reference amino acid sequence will comprise at least twenty (20) contiguous amino acid residues, and may be 30, 40, 50, 100, or more residues. Corrections for increased sequence identity associated with inclusion of gaps in the variant's amino acid sequence can be made by assigning gap penalties. Methods of sequence alignment are well known in the art for both amino acid sequences and for the nucleotide sequences encoding amino acid sequences.

Thus, the determination of percent identity between any two sequences can be accomplished using a mathematical algorithm. One preferred, non-limiting example of a mathematical algorithm utilized for the comparison of sequences is the algorithm of Myers and Miller (1988) CABIOS 4:11-17. Such an algorithm is utilized in the ALIGN program (version 2.0), which is part of the GCG sequence alignment software package. A PAM120 weight residue table, a gap length penalty of 12, and a gap penalty of 4 can be used with the ALIGN program when comparing amino acid sequences. Another preferred, nonlimiting example of a mathematical algorithm for use in comparing two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:403. BLAST nucleotide searches can be performed with the NBLAST program, score=100, wordlength=12, to obtain nucleotide sequences homologous to a nucleotide sequence encoding the polypeptide of interest. BLAST protein searches can be performed with the XBLAST program, score=50, wordlength=3, to obtain amino acid sequences homologous to the polypeptide of interest. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See www.ncbi.nlm.nih.gov. Also see the ALIGN program (Dayhoff (1978) in Atlas of Protein Sequence and Structure 5:Suppl. 3 (National Biomedical Research Foundation, Washington, D.C.) and programs in the Wisconsin Sequence Analysis Package, Version 8 (available from Genetics Computer Group, Madison, Wis.), for example, the GAP program, where default parameters of the programs are utilized.

When considering percentage of amino acid sequence identity, some amino acid residue positions may differ as a result of conservative amino acid substitutions, which do not affect properties of protein function. In these instances, percent sequence identity may be adjusted upwards to account for the similarity in conservatively substituted amino acids. Such adjustments are well known in the art. See, for example, Myers & Miller (1988) Computer Applic. Biol. Sci. 4:11-17.

The angiogenic agents of the invention are formulated into pharmaceutical compositions for use in the methods of the invention. In this manner, a pharmaceutically acceptable carrier may be used in combination with the angiogenic agent and other components in the pharmaceutical composition. By “pharmaceutically acceptable carrier” is intended a carrier or diluent that is conventionally used in the art to facilitate the storage, administration, and/or the desired effect of the therapeutic ingredients. A carrier may also reduce any undesirable side effects of the angiogenic agent. A suitable carrier should be stable, i.e., incapable of reacting with other ingredients in the formulation. It should not produce significant local or systemic adverse effect in recipients at the dosages and concentrations employed for therapy. Such carriers are generally known in the art. Suitable carriers for this invention are those conventionally used large stable macromolecules such as albumin, gelatin, collagen, polysaccharide, monosaccarides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), heparin alginate, and the like. Slow-release carriers, such as hyaluronic acid, may also be suitable. Stabilizers, such as trehalose, thioglycerol, and dithiothreitol (DTT), may also be added. Other acceptable components in the composition include, but are not limited to, pharmaceutically acceptable agents that modify isotonicity including water, saline, salts, sugars, polyols, amino acids, and buffers. Examples of suitable buffers include phosphate, citrate, succinate, acetic acid, and other organic acids or their salts and salts that modify the tonicity such as sodium chloride, sodium phosphate, sodium sulfate, potassium chloride, and can also include the buffers listed above. Further, the agents of the invention may be administered using a patch for slow release. Such formulations may include DMSO.

Preferred pharmaceutical compositions may incorporate buffers having reduced local pain and irritation resulting from injection. Such buffers include, but are not limited to, low phosphate buffers and succinate buffers. The pharmaceutical composition may additionally comprise a solubilizing compound that is capable of enhancing the solubility of an angiogenic agent or variant.

For the purposes of this invention, the pharmaceutical composition comprising the angiogenic agent should be formulated in a unit dosage and in an injectable or infusible form such as solution, suspension, or emulsion. It can also be in the form of lyophilized powder, which can be converted into solution, suspension, or emulsion before administration. The pharmaceutical composition may be sterilized by membrane filtration and stored in unit-dose or multi-dose containers such as sealed vials or ampules.

The method for formulating a pharmaceutical composition is generally known in the art. A thorough discussion of formulation and selection of pharmaceutically acceptable carriers, stabilizers, and isomolytes can be found in Remington's Pharmaceutical Sciences (18^{th} ed.; Mack Pub. Co.: Eaton, Pa., 1990), herein incorporated by reference.

The pharmaceutical compositions of the present invention can also be formulated in a sustained-release form to prolong the presence of the pharmaceutically active agent in the treated patient, generally for longer than one day. Many methods of preparation of a sustained-release formulation are known in the art and are disclosed in Remington's Pharmaceutical Sciences (18^{th} ed.; Mack Pub. Co.: Eaton, Pa., 1990), herein incorporated by reference. Generally, the agent can be entrapped in semipermeable matrices of solid hydrophobic polymers. The matrices can be shaped into films or microcapsules. Examples of such matrices include, but are not limited to, polyesters, copolymers of L-glutamic acid and gamma ethyl-L-glutamate (Sidman et al. (1983) Biopolymers 22:547-556), poly-actides (U.S. Pat. No. 3,773,919 and EP 58,481), polyactate polyglycolate (PLGA), hydrogels (see, for example, Langer et al. (1981) J. Biomed. Mater. Res. 15:167-277; Langer (1982) Chem. Tech. 12:98-105), non-degradable ethylene-vinyl acetate, degradable lactic acid-glycolic acid copolymers such as the Lupron Depots, and poly-D-(−)-3-hydroxybutyric acid (EP 133,988). Suitable microcapsules can also include hydroxymethylcellulose or gelatin-microcapsules and poly-methylmethacylate microcapsules prepared by coacervation techniques or by interfacial polymerization. Microparticles such as heparin alginate beads may also be used. In addition, microemulsions or colloidal drug delivery systems such as liposomes and albumin microspheres, may also be used. See Remington's Pharmaceutical Sciences (18^{th} ed.; Mack Pub. Co.: Eaton, Pa., 1990).

While any angiogenic agent may be utilized in the methods of the invention, of particular interest is FGF. The fibroblast growth factors (FGF) are a family of at least twenty-three structurally related polypeptides (named FGF-1 to FGF-23) that are characterized by a high degree of affinity for proteoglycans, such as heparin. The various FGF molecules range in size from 15 to at least 32.5 kDa, and exhibit a broad range of biological activities in normal and malignant conditions including nerve cell adhesion and differentiation (Schubert et al. (1987) J. Cell Biol. 104:635-643); wound healing (U.S. Pat. No. 5,439,818 (Fiddes)); as mitogens toward many mesodermal and ectodermal cell types, as trophic factors, as differentiation inducing or inhibiting factors (Clements et al. (1993) Oncogene 8:1311-1316); and as an angiogenic factor (Harada (1994) J. Clin. Invest. 94:623-630). Thus, the FGF family is a family of pluripotent growth factors that stimulate to varying extents fibroblasts, smooth muscle cells, epithelial cells, endothelial cells, myocytes, and neuronal cells. FGF-like polypeptides are also contemplated for use in the compositions and methods of the present invention. By “FGF-like” is intended polypeptides that bind FGF receptor 1, particularly receptor 1-C, bind to heparin-like molecules, and have angiogenic activity. By heparin-like molecule is intended heparin, proteoglycans and other polyanionic compounds that bind FGF, that dimerize FGF and that facilitate receptor activation. Of particular interest in the practice of the invention is the FGF designated FGF-2 as well as variants and fragments thereof, which are known in the art. For example, see U.S. Pat. Nos. 5,989,866; 5,925,528; 5,874,254; 5,852,177; 5,817,485; 5,714,458; 5,656,458; 5,604,293; 5,576,288; 5,514,566; 5,482,929; 5,464,943; and 5,439,818.

The FGF, more particularly FGF-2, to be administered can be from any animal species including, but not limited to, avian, canine, bovine, porcine, equine, and human. Generally, the FGF is from a mammalian species, preferably bovine or human in the case of FGF-2. The FGF may be in the native, recombinantly produced, or chemically synthesized forms as outlined below. Where the FGF is FGF-2, it may be the 146 amino acid form, the 153-155 amino acid form, or a mixture thereof depending upon the method of recombinant production. See U.S. Pat. No. 5,143,829, herein incorporated by reference. Further, angiogenically active muteins of the FGF-2 molecule can be used. See, for example, U.S. Pat. Nos. 5,859,208 and 5,852,177, herein incorporated by reference.

In particular, a mammalian fibroblast growth factor of bovine origin, the FGF-2 set forth in SEQ ID NO:2, also known as basic FGF (bFGF), and human FGF-2 (hFGF-2) set forth in SEQ ID NO:4, or biologically active or angiogenically active fragment or mutein thereof, can be utilized in the practice of the invention. The nucleotide sequence encoding the bovine FGF-2 protein is set forth in SEQ ID NO: 1. The nucleotide sequence encoding human FGF-2 is set forth in SEQ ID NO:3. See also, U.S. Pat. No. 5,604,293, herein incorporated by reference. The FGF of the invention may be administered as a unit dose of about 0.2 μg/kg to about 36 μg/kg into one or more vessels of a patient having erectile dysfunction. Such administration improves erectile function in the patient.

Because FGF-2 is a glycosoaminoglycan-(e.g., heparin) binding protein and the presence of a glycosoaminoglycan (also known as a “proteoglycan” or a “mucopolysaccharide”) optimizes activity and area under the curve (AUC), the dosages of FGF-2 of the present invention may be administered within 20 minutes of an intravenous (IV) administration of a glycosoaminoglycan, such as a heparin. Various fractionated and unfractionated heparins, proteoglycans, and sulfated mucopolysaccharides such as chondroitin sulfate can be used in the practice of the invention. Low molecular weight heparins (<10,000 d) and unfractionated (i.e., high molecular weight) heparins (>10,000 d) can be used in the practice of the invention. These molecules can be administered together with the rFGF-2 or within 20 to 30 minutes of administration of the rFGF-2. Heparin is suitably dosed at 20-80 units/kg, and preferably at 40 units/kg.

In one embodiment, the unit dose contains a sufficient amount of FGF-2 ranging from about 0.2 μg/kg about 36 μg/kg. More typically, the systemic unit dose comprises 0.3 mg to 3.5 mg of the FGF-2 of SEQ ID NO:2, or the FGF-2 of SEQ ID NO:4, or biologically active or angiogenically active fragment or mutein thereof. It is recognized that lower doses may be preferable for local delivery. Dosages for local delivery comprising about 0.01 μg to about 500 μg, up to about 3 mg may be used. The unit dose is typically provided in solution or lyophilized form containing the above referenced amount of FGF-2 and an effective amount of one or more pharmaceutically acceptable buffers, stabilizers, and/or other excipients as described elsewhere herein.

The recombinant FGF-2 having the amino acid sequence set forth in SEQ ID NO:2 is made as described in U.S. Pat. No. 5,155,214, entitled “Basic Fibroblast Growth Factor,” which issued on Oct. 13, 1992, and which is incorporated herein by reference in its entirety. As disclosed in the '214 patent, the DNA set forth in SEQ ID NO:1, which encodes a bFGF (hereinafter “FGF-2”) set forth in SEQ ID NO:2, is inserted into a cloning vector, such as pBR322, pMB9, Col E1, pCRI, RP4 or λ-phage, and the cloning vector is used to transform either a eukaryotic or prokaryotic cell, wherein the transformed cell expresses the FGF-2. In one embodiment, the host cell is a yeast cell, such as Saccharomyces cerevisiae. The resulting full-length FGF-2 that is expressed has 146 amino acids as set forth in SEQ ID NO:2. Although the FGF-2 set forth in SEQ ID NO:2 has four cysteines, i.e., at residue positions 25, 69, 87 and 92, there are no internal disulfide linkages. ['214 at col. 6, lines 59-61.] However, in the event that cross-linking occurred under oxidative conditions, it would likely occur between the residues at positions 25 and 69.

The 146-residue mammalian FGF-2 set forth in SEQ ID NO:2, which is of bovine origin, like the corresponding 146-residue human FGF-2 (SEQ ID NO:4), is initially synthesized in vivo as a polypeptide having 155 amino acids (Abraham et al. (1986) EMBO J. 5(10):2523-2528; SEQ ID NO:6 of bovine origin; SEQ ID NO:8 of human origin). When compared to the full-length 155-residue FGF-2 molecules, the 146-residue FGF-2 molecules lack the first nine amino acid residues, Met-Ala-Ala-Gly-Ser-Ile-Thr-Thr-Leu (SEQ ID NO:9), at the N-terminus of the corresponding full-length 155-residue bovine and human FGF-2 molecules (SEQ ID NO:6 and SEQ ID NO:8, respectively). The 155-residue FGF-2 of bovine or human origin, and biologically or angiogenically active variants thereof, can also be used in the compositions and methods of the present invention in the manner described for the bovine and human 146-residue FGF-2 molecules. Again, it is recognized that the 155-residue form may exist as 153-155 residues, or mixtures thereof, depending upon the method of recombinant protein production. The mammalian FGF-2 set forth in SEQ ID NO:2 differs from human FGF-2 set forth in SEQ ID NO:4 in two residue positions. In particular, the amino acids at residue positions 112 and 128 of the mammalian FGF-2 set forth in SEQ ID NO:2 are Ser and Pro, respectively, whereas in human FGF-2 (SEQ ID NO:4), they are Thr and Ser, respectively. For the 155-residue forms, these differences appear at residue positions 121 and 137 of SEQ ID NO:6 (FGF-2 of bovine origin) and SEQ ID NO:8 (FGF-2 of human origin).

The recombinant FGF-2 employed in the present compositions and method was purified to pharmaceutical quality (98% or greater purity) using the techniques described in detail in U.S. Pat. No. 4,956,455, entitled “Bovine Fibroblast Growth Factor,” which issued on Sep. 11, 1990 and which is incorporated herein by reference in its entirety. In particular, the first two steps employed in the purification of the recombinant FGF-2 used in a unit dose of a pharmaceutical composition of the invention are “conventional ion-exchange and reverse phase HPLC purification steps as described previously.” [U.S. Pat. No. 4,956,455, citing to Bolen et al. (1984) Proc. Natl. Acad. Sci. USA 81:5364-5368.] The third step, which the '455 patent refers to as the “key purification step” ['455 at col. 7, lines 5-6], is heparin-SEPHAROSE® affinity chromatography, wherein the strong heparin binding affinity of the FGF-2 is utilized to achieve several thousand-fold purification when eluting at approximately 1.4 M and 1.95 M NaCl ['455 at col. 9, lines 20-25]. Polypeptide homogeneity was confirmed by reverse-phase high pressure liquid chromatography (RP-HPLC). Buffer exchange was achieved by SEPHADEX® G-25(M) gel filtration chromatography.

In addition to the 146-residue FGF-2 set forth in SEQ ID NO:2, the therapeutically active agent in the unit dose of the present invention also comprises an “angiogenically active fragment” or “biologically active fragment” of the FGF-2 set forth in SEQ ID NO:2. By the term “angiogenically or biologically active fragment of the FGF-2 of SEQ ID NO:2” is meant a fragment of FGF-2 that has about 80% of the 146 residues of SEQ ID NO:2 and that retains the angiogenic or biological effect of the FGF-2 of SEQ ID NO:2. This definition of “angiogenically or biologically active fragment” also applies to human FGF-2 set forth in SEQ ID NO:4. An “angiogenically or biologically active “fragment” of the FGF-2 of SEQ ID NO:6 or SEQ ID NO:8 is a fragment of FGF-2 that has about 80% of the 155-residues of SEQ ID NO:6 or SEQ ID NO:8, respectively.

To be active in promoting endothelial cell proliferation and activity and in generally promoting angiogenesis, the FGF-2 fragment should have two cell binding sites and at least one of the two heparin binding sites. The two putative cell binding sites of the analogous 146-residue human FGF-2 (hFGF-2; SEQ ID NO:4) occur at about residue positions 36-39 and about 77-81 thereof. See Yoshida et al. (1987) Proc. Natl. Aca. Sci. USA 84:7305-7309. The two putative heparin binding sites of hFGF-2 occur at about residue positions 18-22 and about 107-111 thereof. See Yoshida et al. (1987) supra. Given the substantial similarity between the amino acid sequences for human FGF-2 (hFGF-2) and bovine FGF-2 (bFGF-2), it is expected that the cell binding sites for bFGF-2 (SEQ ID NO:2) are also at about residue positions 36-39 and about 77-81 thereof, and that the heparin binding sites are at about residue positions 18-22 and about 107-111 thereof. The additional 9 residues of the 155-residue form do not affect the relative positions of these binding sites with respect to residues 1-146 shown in SEQ ID NO:2 (FGF-2 of bovine origin) or SEQ ID NO:4 (FGF-2 of human origin). Thus, for the 155-residue form of human FGF-2 (SEQ ID NO:8), the two putative cell binding sites occur at about residue positions 45-48 and about 86-90 thereof, and the two putative heparin binding sites occur at about residue positions 27-31 and about 116-120 thereof. Again, given the substantial similarity between the 155-residue bovine and human proteins, it is expected that the two putative cell binding sites are at about residue positions 45-48 and about 86-90, and the two putative heparin binding sites are at about residue positions 27-31 and about 116-120 of the 155-residue bovine FGF-2 (SEQ ID NO:6). Consistent with the above, it is well known in the art that N-terminal truncations of the FGF-2 of SEQ ID NO:2 do not eliminate its angiogenic activity in cows. In particular, the art discloses several naturally occurring and biologically active fragments of the FGF-2 that have N-terminal truncations relative to the FGF-2 of SEQ ID NO:2. An active and truncated bFGF-2 having residues 12-146 of SEQ ID NO:2 was found in bovine liver and another active and truncated bFGF-2, having residues 16-146 of SEQ ID NO:2 was found in the bovine kidney, adrenal glands, and testes. (See U.S. Pat. No. 5,155,214 at col. 6, lines 41-46, citing to Ueno, et al. (1986) Biochem. Biophys. Res. Comm. 138:580-588.) Likewise, other fragments of the bFGF-2 of SEQ ID NO:2 that are known to have FGF activity are FGF-2 (24-120)-OH and FGF-2 (30-110)-NH₂. [U.S. Pat. No. 5,155,214 at col. 6, lines 48-52.] These latter fragments retain both of the cell binding portions of FGF-2 (SEQ ID NO:2) and one of the heparin binding segments (residues 107-111). Accordingly, the biologically active fragments of a mammalian FGF typically encompass those terminally truncated fragments of an FGF-2 that have at least residues that correspond to residues 30-110 of rFGF-2 of SEQ ID NO:2; more typically, at least residues that correspond to residues 18-146 of rFGF-2 of SEQ ID NO:2.

It is recognized that other synthetic peptides based on native FGF sequences may be used as long as these peptides bind FGF receptors. Additionally hybrid FGF molecules may be constructed comprising peptides from different native sequences as well as combinations of native and synthetic sequences. Again, the hybrid molecules will retain the ability to bind with FGF receptors.

The unit dose of the present invention also comprises an “angiogenically active mutein” of the FGF-2 of SEQ ID NO:2, SEQ ID NO:4, SEQ ID NO:6, or SEQ ID NO:8. By the term “angiogenically active mutein” is intended a mutated form of the FGF-2 of SEQ ID NO:2, SEQ ID NO:4, SEQ ID NO:6, or SEQ ID NO:8 that structurally retains at least 80%, preferably 90%, of the 146 residues of the FGF-2 sequence shown in SEQ ID NO: 2, the 146 residues of human FGF-2 sequence shown in SEQ ID NO:4, the 155 residues of the FGF-2 sequence shown in SEQ ID NO:6, or the 155 residues of the FGF-2 sequence shown in SEQ ID NO:8, respectively, in their respective positions, and that functionally retains the angiogenic activity of the respective unmutated form of FGF-2. Preferably, the mutations are “conservative substitutions” using L-amino acids, wherein one amino acid is replaced by another biologically similar amino acid. Examples of conservative substitutions include the substitution of one hydrophobic residue such as Ile, Val, Leu, Pro, or Gly for another, or the substitution of one polar residue for another, such as between Arg and Lys, between Glu and Asp, or between Gln and Asn, and the like. Generally, the charged amino acids are considered interchangeable with one another. However, to make the substitution more conservative, one takes into account both the size and the likeness of the charge, if any, on the side chain. Suitable substitutions include the substitution of serine for one or both of the cysteines at residue positions 87 and 92 of SEQ ID NO:2 or SEQ ID NO:4, or at residue positions 96 and 101 of SEQ ID NO:6 or SEQ ID NO:8, which are not involved in disulfide formation. Other suitable substitutions include any substitution wherein at least one constituent cysteine is replaced by another amino acid so that the mutein has greater stability under acidic conditions, see for example U.S. Pat. No. 5,852,177, which is herein incorporated by reference. One such substitution is the replacement of cysteine residues with neutral amino acids such as for example: glycine, valine, alanine, leucine, isoleucine, tyrosine, phenylalanine, histidine, tryptophan, serine, threonine, and methionine (U.S. Pat. No. 5,852,177). Preferably, substitutions are introduced at the FGF-2 N-terminus, which is not associated with angiogenic activity. However, as discussed above, conservative substitutions are suitable for introduction throughout the molecule.

One skilled in the art, using well-known techniques, is able to make one or more point mutations in the DNA of SEQ ID NO: 1, SEQ ID NO:3, SEQ ID NO:5, or SEQ ID NO:7 to obtain expression of an FGF-2 polypeptide mutein (or fragment of a mutein) having angiogenic activity for use within the unit dose, compositions, and methods of the present invention. To prepare a biologically active mutein of the FGF-2 of SEQ ID NO:2, SEQ ID NO:4, SEQ ID NO:6, or SEQ ID NO:8, one uses standard techniques for site-directed mutagenesis, as known in the art and/or as taught in Gilman et al. (1979) Gene 8:81 or Roberts et al. (1987) Nature 328:731, to introduce one or more point mutations into the cDNA of SEQ ID NO: 1, SEQ ID NO:3, SEQ ID NO:5, or SEQ ID NO:7 that encodes the FGF-2 of SEQ ID NO:2, SEQ ID NO:4, SEQ ID NO:6, or SEQ ID NO:8, respectively.

Pharmaceutical compositions of the invention comprise a biologically effective dose of a mammalian FGF-2 of SEQ ID NO:2, SEQ ID NO:4, SEQ ID NO:6, or SEQ ID NO:8, or a biologically active fragment or mutein thereof, and a pharmaceutically acceptable carrier. Typically, the safe and biologically effective dose of the pharmaceutical composition of the present invention is in a form and a size suitable for administration to a human patient and comprises (i) 0.2 μg/kg to 36 μg/kg of an FGF-2 of SEQ ID NO:2 or a biologically active fragment or mutein thereof, (ii) and a pharmaceutically acceptable carrier. In other embodiments, the safe and biologically effective dose comprises 0.005 μg/kg to 5 μg/kg, 0.1 μg/kg to 2 μg/kg or 2 μg/kg to 36 μg/kg of the FGF-2 of SEQ ID NO:2, SEQ ID NO:4, SEQ ID NO:6, or SEQ ID NO:8, or a biologically active fragment or mutein thereof, and a pharmaceutically acceptable carrier.

A typical pharmaceutical composition comprises 0.001 to 10 mg/ml, more typically 0.03 to 0.5 mg/ml, of FGF-2, more particularly recombinant FGF-2 (rFGF-2), having the sequence set forth in SEQ ID NO:2, or in SEQ ID NO:4, or a biologically active fragment or mutein thereof, 10 mM thioglycerol, 135 mM NaCl, 10 mM Na citrate, and 1 mM EDTA, pH 5.0. A suitable diluent or flushing agent for the above-described composition is any of the above-described carriers. Typically, the diluent is the carrier solution itself comprising 10 mM thioglycerol, 135 mM NaCl, 10 MM Na citrate, and 1 mM EDTA, pH 5.0. The rFGF-2 of SEQ ID NO:2 or a biologically active fragment or mutein thereof is unstable for long periods of time in liquid form. To maximize stability and shelf life, the pharmaceutical composition of the present invention comprising an effective amount of rFGF-2 or a biologically fragment or mutein thereof, in a pharmaceutically acceptable aqueous carrier should be stored frozen at −60° C. When thawed, the solution is stable for 6 months at refrigerated conditions. A typical unit dose would comprise about 5-10 ml of the above described composition having 1.5-8 mg of FGF-2 of SEQ ID NO:2, or SEQ ID NO:4.

In another embodiment, the pharmaceutical composition comprises a unit dose of FGF-2 of SEQ ID NO:2, SEQ ID NO:4, or a biologically active fragment or mutein thereof in lyophilized (freeze-dried) form. In this form, the unit dose of FGF-2 would be capable of being stored at refrigerated temperatures for substantially longer than 6 months without loss of therapeutic effectiveness. Lyophilization is accomplished by the rapid freeze drying under reduced pressure of a plurality of vials, each containing a unit dose of the FGF-2 of the present invention therein. Lyophilizers, which perform the above described lyophilization, are commercially available and readily operable by those skilled in the art. Prior to administration to a patient, the lyophilized product is reconstituted to a known concentration, preferably in its own vial, with an appropriate sterile aqueous diluent, typically 0.9% (or less) sterile saline solution, or a compatible sterile buffer, or even sterile deionized water. Depending upon the weight of the patient in kg, a single dose comprising from 0.2 μg/kg to 36 μg/kg of the FGF-2 of SEQ ID NO:2, the FGF-2 of SEQ ID NO:4, or a biologically active fragment or mutein thereof is withdrawn from the vial as reconstituted product for administration to the patient. Thus, an average 70 kg man that is being dosed at 24 μg/kg, would have a sufficient volume of the reconstituted product withdrawn from the vial to receive an intra-arterial or transmural infusion of (70 kg×24 μg/kg) 1680 μg (i.e., 1.680 mg).

The pharmaceutical composition in solution form is generally administered by infusing the unit dose substantially continuously over a period of about 10 to about 30 minutes, although it is recognized that the composition may be administered over a longer period of time. When the composition is administered into more than one blood vessel, typically, a portion (e.g., one half) of the unit dose is administered in a first vessel followed by administration into a second secondary vessel. Using the above-described repositioning procedure, portions of the unit dose may be administered to a plurality of vessels until the entire unit dose has been administered. After administration, the catheter is withdrawn using conventional protocols known in the art. Signs of angiogenesis are apparent in a matter of days following intra-arterial or transmural administration of the unit dose. Therapeutic benefit is seen as early as two weeks following the FGF-2 administration.

For information on pharmacokinetics and metabolism of the FGF formulation, see copending U.S. application Ser. No. 09/385,114, entitled “Angiogenically Effective Unit Dose of FGF-2 and Method of Use,” herein incorporated by reference.

The following examples are offered by way of illustration and not by way of limitation.

### EXPERIMENTAL

### EXAMPLE 1

### Unit Dose of rFGF-2 Employed in a Phase I Clinical Trial

The rFGF-2 of SEQ ID NO:2 was formulated as a unit dose and pharmaceutical composition. The various formulations are described below.

The rFGF-2 unit dose was provided as a liquid in 3 cc type I glass vials with a laminated gray butyl rubber stopper and red flip-off overseal. The rFGF-2 unit dose contained 1.2 ml of 0.3 mg/ml rFGF-2 of SEQ ID NO:2 in 10 mM sodium citrate, 10 mM monothioglycerol, 1 mM disodium dihydrate EDTA (molecular weight 372.2), 135 mM sodium chloride, pH 5.0. Thus, in absolute terms, each vial (and unit dose) contained 0.36 mg rFGF-2. The vials containing the unit dose in liquid form were stored at 2° to 8° C.

The rFGF diluent was supplied in 5 cc type I glass vials with a laminated gray butyl rubber stopper and red flip-off overseal. The rFGF-2 diluent contains 10 mM sodium citrate, 10 mM monothioglycerol, 135 mM sodium chloride, pH 5.0. Each vial contained 5.2 ml of rFGF-2 diluent solution that was stored at 2° to 8° C.

The rFGF-2 pharmaceutical composition that was infused was prepared by diluting the rFGF-2 unit dose with the rFGF diluent such that the infusion volume is 10 ml. In order to keep the EDTA concentration below the limit of 100 μg/ml, the total infusion volume was increased to 20 ml when proportionately higher absolute amounts of FGF-2 were administered to patients with high body weights.

### EXAMPLE 2

### Clinical Study of Recombinant FGF-2 Administered to Humans

The rFGF-2 of SEQ ID NO:2 was formulated as a unit dose and pharmaceutical composition for administration to humans. The various formulations are described below.

The rFGF-2 unit dose was prepared as a liquid in 5 cc type I glass vials with a laminated gray butyl rubber stopper and red flip-off overseal. The rFGF-2 formulation contains 0.3 mg/ml rFGF-2 of SEQ ID NO:2 in 10 mM sodium citrate, 10 mM monothioglycerol, 0.3 mM disodium dihydrate EDTA (molecular weight 372.2), 135 mM sodium chloride, pH 5.0. Each vial contained 3.7 ml of rFGF-2 drug product solution (1.11 mg rFGF-2 per vial). The resulting unit dose in liquid form was stored at less than 60° C. The above described unit dose is diluted with the “rFGF-2 placebo.”

The rFGF placebo is supplied as a clear colorless liquid in 5 cc type I glass vials with a laminated gray butyl rubber stopper and red flip-off overseal. The rFGF-2 placebo is indistinguishable in appearance from the drug product and has the following formulation: 10 mM sodium citrate, 10 mM monothioglycerol, 0.3 mM disodium dihydrate EDTA (molecular weight 372.2), 135 mM sodium chloride, pH 5.0. Each vial contains 5.2 ml of rFGF-2 placebo solution. The rFGF-2 placebo is stored at 2° to 8° C.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

That which is claimed:

1. A method for treating or preventing erectile dysfunction in a patient, said method comprising administering to said patient a therapeutically effective amount of a growth factor, wherein said growth factor is selected from the group consisting of FGF, EGF, PDGF, VEGF, and TGF.

2. The method of claim 1, wherein said growth factor is administered to one or more blood vessels in said patient.

3. The method of claim 2, wherein said growth factor is FGF.

4. The method of claim 3, wherein said FGF is FGF-2.

5. The method of claim 4, wherein said FGF-2 is a recombinant molecule.

6. The method of claim 5, wherein said FGF-2 comprises the sequence set forth in SEQ ID NO:2, SEQ ID NO:4, SEQ ID NO:6, SEQ ID NO:8, or a biologically active fragment or mutein thereof.

7. The method of claim 6, wherein said therapeutically effective amount of said FGF-2 or said biologically active fragment or mutein thereof is about 0.008 mg to about 5.1 mg.

8. The method of claim 7, wherein said therapeutically effective amount of said FGF-2 or said biologically active fragment or mutein thereof is about 0.3 mg to about 3.5 mg.

9. The method of claim 7, wherein said therapeutically effective amount of said FGF-2 or said biologically active fragment or mutein thereof is about 0.008 mg to about 5.1 mg.

10. The method of claim 7, wherein said therapeutically effective amount of said FGF-2 or said biologically active fragment or mutein thereof is about 0.008 mg to about 5.1 mg.

11. The method of claim 6, wherein said therapeutically effective amount of said FGF-2 or said biologically active fragment or mutein thereof is about 0.2 μg/kg to about 36 μg/kg.

12. The method of claim 11, wherein said therapeutically effective amount of said FGF-2 or said biologically active fragment or mutein thereof is about 0.2 μg/kg to about 2 μg/kg.

13. The method of claim 11, wherein said therapeutically effective amount of said FGF-2 or said biologically active fragment or mutein thereof is about 2 μg/kg to about 20 μg/kg.

14. The method of claim 11, wherein said therapeutically effective amount of said FGF-2 or said biologically active fragment or mutein thereof is about 20 μg/kg to about 36 μg/kg.

15. The method of claim 1, wherein said growth factor is administered by transmural delivery.

16. A method for improving erectile function in a mammal, said method comprising delivering at a target site in said mammal in a therapeutically effective amount a pharmaceutical composition, said composition comprising a growth factor selected from the group consisting of FGF, EGF, PDGF, VEGF, and TGF and a pharmaceutically acceptable carrier.

17. The method of claim 16, wherein said growth factor is administered to one or more blood vessels in said patient.

18. The method of claim 17, wherein said growth factor is FGF.

19. The method of claim 18, wherein said FGF is FGF-2.

20. The method of claim 19, wherein said FGF-2 is a recombinant molecule.

21. The method of claim 19, wherein said FGF-2 comprises the sequence set forth in SEQ ID NO:2, SEQ ID NO:4, SEQ ID NO:6, SEQ ID NO:8, or a biologically active fragment or mutein thereof.

22. The method of claim 21, wherein said therapeutically effective amount of said FGF-2 or said biologically active fragment or mutein thereof is about 0.008 mg to about 5.1 mg.

23. The method of claim 22, wherein said therapeutically effective amount of said FGF-2 or said biologically active fragment or mutein thereof is about 0.3 mg to about 3.5 mg.

24. The method of claim 22, wherein said therapeutically effective amount of said FGF-2 or said biologically active fragment or mutein thereof is about 0.008 mg to about 5.1 mg.

25. The method of claim 22, wherein said therapeutically effective amount of said FGF-2 or said biologically active fragment or mutein thereof is about 0.008 mg to about 5.1 mg.

26. The method of claim 21, wherein said therapeutically effective amount of said FGF-2 or said biologically active fragment or mutein thereof is about 0.2 μg/kg to about 36 μg/kg.

27. The method of claim 26, wherein said therapeutically effective amount of said FGF-2 or said biologically active fragment or mutein thereof is about 0.02 μg/kg to about 2 μg/kg.

28. The method of claim 26, wherein said therapeutically effective amount of said FGF-2 or said biologically active fragment or mutein thereof is about 2 μg/kg to about 20 μg/kg.

29. The method of claim 26, wherein said therapeutically effective amount of said FGF-2 or said biologically active fragment or mutein thereof is about 20 μg/kg to about 36 μg/kg.


SEQUENCE LISTING (9 sequences)

SEQ ID NO: 1 - DNA, 441 bp, Bos taurus
  CDS (1)...(441)
ccagccctaccagaagatggggggtccggggccttcccaccagggcacttcaaagatccaaaacgactatattgtaaaaacggggggttcttcctacgaatccacccagatgggcgagtagatggggtacgagaaaaatccgatccacacatcaaactacaactacaagccgaagaacgaggggtagtatccatcaaaggggtatgtgccaaccgatatctagccatgaaagaagatgggcgactactagcctccaaatgtgtaaccgatgaatgtttcttcttcgaacgactagaatccaacaactataacacctatcgatcccgaaaatattcctcctggtatgtagccctaaaacgaaccgggcaatataaactagggccaaaaaccgggccagggcaaaaagccatcctattcctaccaatgtccgccaaatcctaa

SEQ ID NO: 2 - protein, 146 aa, Bos taurus
PALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKS

SEQ ID NO: 3 - DNA, 441 bp, Homo sapiens
  CDS (1)...(441)
cccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctga

SEQ ID NO: 4 - protein, 146 aa, Homo sapiens
PALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

SEQ ID NO: 5 - DNA, 468 bp, Bos taurus
  CDS (1)...(468)
atggcagccgggagcatcaccacgctgccagccctaccagaagatggggggtccggggccttcccaccagggcacttcaaagatccaaaacgactatattgtaaaaacggggggttcttcctacgaatccacccagatgggcgagtagatggggtacgagaaaaatccgatccacacatcaaactacaactacaagccgaagaacgaggggtagtatccatcaaaggggtatgtgccaaccgatatctagccatgaaagaagatgggcgactactagcctccaaatgtgtaaccgatgaatgtttcttcttcgaacgactagaatccaacaactataacacctatcgatcccgaaaatattcctcctggtatgtagccctaaaacgaaccgggcaatataaactagggccaaaaaccgggccagggcaaaaagccatcctattcctaccaatgtccgccaaatcctaa

SEQ ID NO: 6 - protein, 155 aa, Bos taurus
MAAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYSSWYVALKRTGQYKLGPKTGPGQKAILFLPMSAKS

SEQ ID NO: 7 - DNA, 474 bp, Homo sapiens
  CDS (1)...(468)
atggcagccgggagcatcaccacgctgcccgccttgcccgaggatggcggcagcggcgccttcccgcccggccacttcaaggaccccaagcggctgtactgcaaaaacgggggcttcttcctgcgcatccaccccgacggccgagttgacggggtccgggagaagagcgaccctcacatcaagctacaacttcaagcagaagagagaggagttgtgtctatcaaaggagtgtgtgctaaccgttacctggctatgaaggaagatggaagattactggcttctaaatgtgttacggatgagtgtttcttttttgaacgattggaatctaataactacaatacttaccggtcaaggaaatacaccagttggtatgtggcactgaaacgaactgggcagtataaacttggatccaaaacaggacctgggcagaaagctatactttttcttccaatgtctgctaagagctgattttaa

SEQ ID NO: 8 - protein, 155 aa, Homo sapiens [sequence omitted]

SEQ ID NO: 9 - protein, 9 aa, Bos taurus
MAAGSITTL